(19) United States
(12) Patent Application Publication
(10) Pub. No.: US 2019/0011434 A1
(43) Pub. Date: Jan. 10, 2019

(54) METHODS TO DETERMINE CANCER TREATMENT USING ROBOTIC HIGH-THROUGHPUT DRUG SENSITIVITY TESTING

(71) Applicant: AntiCancer, Inc., San Diego, CA (US)

(72) Inventor: Robert M. Hoffman, La Jolla, CA (US)

(21) Appl. No.: 16/029,189

(22) Filed: Jul. 6, 2018

Related U.S. Application Data
(60) Provisional application No. 62/529,351, filed on Jul. 6, 2017.

(51) Int. Cl. G01N 33/50; G01N 35/00

(52) U.S. Cl. CPC G01N 33/5011; G01N 35/0099

(57) ABSTRACT

The present invention describes an in vitro test for drug sensitivity for each cancer patient that is performed with a patient's tumor tissue obtained by surgery or biopsy on an automated tissue processor using multi-well tissue-culture plates containing approximately 1 mm³ tumor tissue culture medium and cancer drug. The present invention will accurately identify both effective and in effective drugs for each patient.

### CROSS REFERENCE TO RELATED APPLICATION

This application claims priority to U.S. (Provisional) Patent Application to Hoffman, entitled “HIGH-TROUGHPUT DRUG-SENSITIVITY TESTING OF CANCER-PATENTS TUMORS EX-VIVO USING A ROBOTIC TISSUE HANDLER,” application No. 62/529,351, filed Jul. 6, 2017, now pending, the disclosure of which is hereby incorporated entirely herein by reference.

### BACKGROUND OF THE INVENTION

### Technical Field

This invention relates to methods of personalized drug therapy for cancer. In particular, the invention relates to methods used to predict a patient's response to chemotherapy by exposing three-dimensional cell cultures derived from the patient's tumor to chemotherapeutic agents.

### State of the Art

The first documented maintenance of tissues out of the body was in 1885 by Wilhelm Roux. Roux maintained the medullary plate of an embryonic chicken in a warm saline solution for several days. This experiment established the principle that tissues could live outside the body. At the end of 1911 and at the beginning of 1912, new techniques were developed for tissue culture by Carrel. Fragments of connective tissue and beating heart were maintained in vitro for more than 2 months. Carrel transferred (passed) the cultures from medium to medium in order to maintain them long term. The overriding consideration in all tissue culture techniques is the need to avoid bacterial contamination of the cultures. The situation was revolutionized by the introduction of antibiotics in the 1940s. In 1923, Carrel introduced the first practical cell culture flask. The flask had good optical properties and a long sloping neck, which prevented contaminants from entering this flask. These flasks allowed the plasma clots to be submerged in a much larger volume of medium than in a hanging drop culture. It was easy to add new medium to the flask.

### The Beginning of Modern Cell Culture

Earle was among the first to establish cell lines that could grow indefinitely, including the L-cell line. HeLa cells were derived from a human cervical tumor by George Gey. Defined cell culture media were first developed by Earle and Ham. Eagle developed a medium with over 25 ingredients. These defined media had to be supplemented with serum such as fetal bovine so that cells could proliferate.

### Histoculture

Histocultures use growth medium with a sponge-gel support that enables 3-dimensional growth of cells and tissues. Histocultures maintain their in vivo-like phenotype in contrast to monolayer cultures or cultures on an extra-cellular matrix coating such as “Matrigel.” By 1951, monolayer cell cultures, in which cells grow as ‘sheets’ on the surfaces of glass or plastic, had become the predominant culture technique and paradigm. Monolayer culture in vitro, however, is not suitable for the study of tissues.

Histocultures can be made from many types of tissues, both cancer and normal. The unit of cultured tissue is ˜1 mm³, readily allowing the diffusion of culture medium nutrients and oxygen into the tissue, obviating the need for a circulatory system. Fragments of tissue can be placed on collagen sponge gels that were developed by Leighton, which are hydrated by culture medium. Placing cells in histoculture enables them to form 3-dimensional structures. Because of its architectural resemblance to native tissue, three-dimensional (3D) histoculture represents a unique in vivo-like model for investigating crucial events in tumor biology, such as drug response, tumor cell migration, invasion, metastasis, immune response and antigen expression. Skin biology and hair growth, and stem cell differentiation can also be investigated in histoculture. Histocultures maintain their in vivo phenotype, including gene expression, in contrast to monolayer cultures.

Leighton made a number of important early observations on the advantages of histoculture; for example, when C3HBA mouse mammary adenocarcinoma cells were grown on sponge-matrix histoculture, he found that the cells aggregated in a manner similar to that in the original tumor. Distinct structures were formed within the tumors such as lumina and stromal elements, with some of the glandular structures similar to the original tumor. Leighton was also able to grow normal tissues such as chick-embryo liver in the sponge-matrix cultures, and he observed that the epithelial cells proliferated and formed glandular structures. On the other hand, when Leighton cultured hepatoma cells in sponge-matrix culture, they behaved differently from the normal liver cells and grew in a loosely packed arrangement as opposed to normal liver cells. It is important to note that, although stromal tissue is present and functional in sponge-gel histoculture of tumors, the fibroblasts are relatively quiescent and do not dominate these cultures as they would in monolayer culture.

Tumors in histoculture, including human tumors, have a drug sensitivity pattern similar to the pattern in the donor patient. In monolayer culture, cancer cells can become artificially sensitive to drugs that does not occur in histoculture. Gene expression, such as coding for cell-surface proteins, is in vivo-like in histoculture, unlike monolayer culture where many genes are no longer expressed, perhaps due to the inability of cells to acquire their normal shape (1).

### DISCLOSURE OF EMBODIMENTS OF THE INVENTION

Embodiments of the present invention include methods for enhancing bacterial targeting of tumors. The foregoing and other features and advantages of the invention will be apparent to those of ordinary skill in the art from the following more particular description of the invention and the accompanying drawings.

Disclosed is a method for determining cancer treatment comprising steps obtaining cells from a tumor of a patient with a cancer; establishing a three-dimensional in-vitro histoculture of the cells; adding a drug to the histoculture; and measuring a response of the histoculture to the drug.

In some embodiments, the drug is mitomycin C. In some embodiments, the drug is doxorubicin. In some embodiments, the drug is 5-flourouracil. In some embodiments, the drug is cisplatin.

In some embodiments, the cancer is gastric cancer. In some embodiments, the cancer is breast cancer. In some embodiments, the cancer is colorectal cancer. In some embodiments, the cancer is epithelial ovarian cancer. In some embodiments, the cancer is squamous cell head and neck cancer.

In some embodiments, the establishing, adding, and measuring steps are performed by a robotic tissue handler.

Disclosed is a robotic tissue handler histoculture drug response assay comprising steps robotically obtaining a plurality of tissue cores from a tumor of a patient with a cancer; placing one each of the plurality of tissue cores into each of a corresponding plurality of wells disposed on a controlled moving stage; adding a mixture of a drug and a culture medium to each of the plurality of wells incubating the plurality of wells; removing the mixture from each of the plurality of wells without removing the tissue cores; adding a cell viability measuring solution to each of the plurality of wells; reading cell viability of the plurality of tissue cores; and correlating the cell viability with a resistance to the drug with an accuracy of at least about seventy-five percent (75%).

In some embodiments, the drug is selected from the group of drugs consisting of mitomycin C, doxorubicin, 5-flourouracil, and cisplatin.

In some embodiments, the cancer is gastric cancer. In some embodiments, the cancer is breast cancer. In some embodiments, the cancer is colorectal cancer. In some embodiments, the cancer is epithelial ovarian cancer. In some embodiments, the cancer is squamous cell head and neck cancer. In some embodiments, the cancer is a sarcoma.

### BRIEF DESCRIPTION OF THE DRAWINGS

FIG. 1 is a flowchart showing steps of method for determining cancer treatment; and

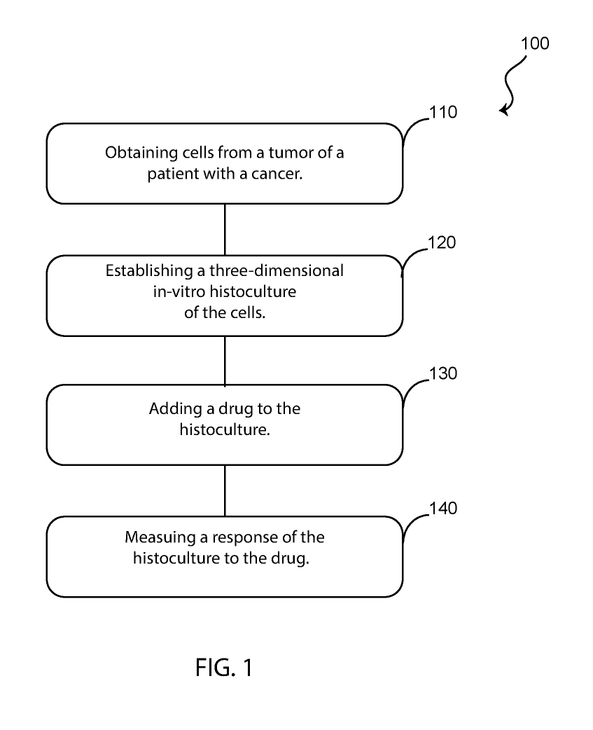

FIG. 2 is a flowchart showing steps of a robotic tissue handler histoculture drug response assay.

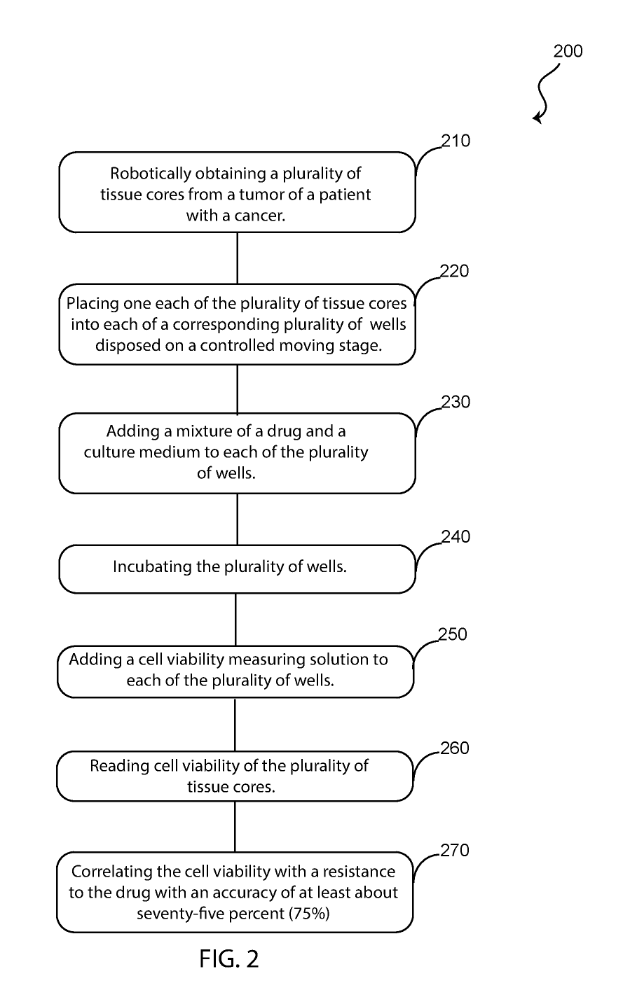

### DETAILED DESCRIPTION OF EMBODIMENTS OF THE INVENTION

As mentioned herein above, the disclosed invention relates to methods used to predict a patient's response to chemotherapy by exposing three-dimensional cell cultures derived from the patient's tumor to chemotherapeutic agents.

We previously developed a sponge-gel-supported culture system for growth of human tumors. In vitro tests of cell sensitivity to drugs that indicates in vivo response is an important need in cancer therapy and cancer drug development. This three-dimensional culture system is general and grows tumors at high frequency directly from surgery or biopsy that maintain important in vivo properties in vitro, including tissue architecture. With autoradiographic techniques measuring cellular DNA synthesis the drug responses of individual cells within the tissue structure of in vitro-grown tumors can be accomplished. Twenty tumor classes, including all the major ones, have been measured in toto at greater than 50% frequency. Quantitative and qualitative results show increasing cell kill with rising cytotoxic drug concentration, differential drug sensitivities of multiple cell types within individual cultured tumors, differential sensitivities of a series of tumors of the same histopathological classification to a single drug, differential sensitivities of individual tumors to a series of drugs, and sensitivity patterns of various tumor types similar to the sensitivities found in vivo. Therefore, the results indicate that potentially important therapeutic data can be obtained from tumor specimens growing in vitro for the individual cancer patient as well as for rational and relevant screening for new agents active against human solid tumors (5). This assay was subsequently termed the histoculture drug response assay (HDRA).

The 3-(4,5-dimethylthiazol-2-yl)-2,5-diphenyl-2H tetrazolium bromide (MTT) end point was applied to the Hoffman assay in an attempt to increase in vitro-in vivo correlation. The chemosensitivities of 16 human tumor lines were determined in vitro by the histoculture drug response assay, and retrospectively correlated to their in vivo chemosensitivity as xenografts in nude mice. The in vitro test was considered to be positive if tumor-cell MTT reduction activity was lowered by more than 50%. The cutoff drug concentrations to determine sensitivity in vitro were determined for mitomycin C, doxorubicin, 5-fluorouracil and cisplatin. Using these cutoff drug concentrations in vitro we found, as a function of time of exposure, a strong correlation between serum drug concentrations found in nude mice given maximum tolerated doses and drug concentrations found in the histoculture media in vitro, thereby establishing a relationship between the amounts of drugs to which tumors were exposed in vivo and in vitro. The overall correlation rate of the efficacy results of the drug-response assay to in vivo chemosensitivities was 89.8%, with 90.0% true-positive and 89.7% true-negative rates, 81.7% sensitivity and 94.6% specificity, thereby indicating potential clinical use for tumor histoculture with the MTT end point (6).

In order to evaluate the HDRA with the 3-(4, 5-dimethylthiazol-2-yl)-2,5-diphenyltetrazolium bromide end point for clinical use, chemosensitivity to mitomycin C, doxorubicin, 5-fluorouracil, and cisplatin of 107 advanced gastric and 109 advanced colorectal cancers was determined in vitro in a correlative clinical trial. Two hundred eight (96.3%) of 216 of the patient specimens were evaluable in the HDRA. Thirty-eight patients with remaining measurable lesions after surgery were evaluable for comparison of the effects of chemotherapy in the HDRA with clinical outcome. Their overall response in the HDRA to all four drugs correlated to published historical data. Twenty-nine patients were treated with drugs shown to be ineffective in the HDRA, and all 29 cases showed clinical chemoresistance. In nine patients treated with drugs shown to be effective in the HDRA, six showed clinical chemoresponse and three showed arrest of disease progression. The correlation rate of the assay to clinical drug-sensitivity response was thus calculated to be 92.1% (35/38), with 100% (29/29) true-negative and 66.7% (6/9) true-positive rates, 100% (6/6) sensitivity, and 90.6% (29/32) specificity. Thirty-two patients with stage III and IV gastric cancer without remaining measurable tumor lesions after surgery were treated with mitomycin C and a fluoropyrimidine adjuvantly. The survival rate of 10 patients whose tumors were sensitive to either mitomycin C and/or 5-fluorouracil in the assay was significantly (P<0.005) better than that of 22 patients whose tumors were shown to be insensitive to both drugs. Twenty-nine patients with stage III and IV colorectal cancer without remaining measurable tumor lesions after surgery were treated with fluoropyrimidines adjuvantly. The recurrence-free survival rate of 7 patients whose tumors were sensitive to 5-fluorouracil in the assay was significantly (P<0.05) better than that of 22 patients whose tumors were insensitive. Thus the HDRA with the 3-(4, 5-dimethylthiazol-2-yl)-2,5-diphenyltetrazolium bromide end point is of clinical value to choose optimal chemotherapy for response as well as for survival (7).

Subsequently, 215 patients with gastric cancer from 45 medical centers were tested with the HDRA in a blinded study after resection of the primary lesion. One hundred sixty-eight patients received at least 20 mg/m2 of mitomycin C and a minimum of 30 g UFT, a mixture of tegafur and uracil at a molar ratio of 1:4, thereby making them eligible for the study. Of these cases 128 were evaluable by the HDRA. The evaluable patient tumors were tested by the HDRA with the [3H]thymidine incorporation end point measured by microautoradiography to be drug “sensitive” or “resistant.” The in vitro conditions for distinguishing sensitivity and resistance that matched the response rates for historical controls for gastric carcinoma were 90% inhibition rate and 0.12 microgram/ml for mitomycin C and 70% inhibition rate and 1 microgram/ml for 5-fluorouracil, respectively. Most importantly in the blinded study, the overall and disease-free survival rates of the HDRA-sensitive group were found to be significantly higher than those of the HDRA-resistant group tested under the above conditions. The data further indicate the importance of three-dimensional tumor culture for obtaining accurate clinical information. The results demonstrate that the HDRA response correlates to patient survival, which suggests the potential of the HDRA to contribute to patient survival in gastric cancer when used prospectively (8).

Subsequently, the HDRA was tested for clinical correlation with head and neck cancer. Cisplatinum (CDDP) sensitivity was compared in the HDRA with patient donors. The criterion for in vitro sensitivity to cisplatin was an 84% or greater inhibition by cisplatin of the number of tritiated thymidine-incorporating cells of the histocultured tumors compared with untreated control culture preparations, as measured by means of histologic autoradiography. Comparisons were made with clinical responses, ie, complete response, partial response, or no response. The study was carried out in patients with head and neck cancers and comprised 21 patients with squamous-cell carcinoma, three patients with other carcinomas, and two patients with sarcoma. Ten of 12 patients with in vitro-sensitive tumors had either complete or partial response clinically. The overall accuracy of the SSHDRA was 74% in this correlative clinical trial; the predictive-positive value was 83%, the sensitivity was 71%, and the specificity was 78%. Seven of 11 patients with in vitro-resistant tumors demonstrated no response for a predictive-negative value of 64%. We conclude that the SSHDRA shows a high correlation for tumors that demonstrate both in vivo drug resistance and sensitivity. The in vitro-like maintenance of three-dimensional tissue architecture of the tumors in histoculture probably contributes to high clinical predictivity of drug response of the SSHDRA. The data support further comparisons to determine the clinical usefulness of the SSHDRA for identifying complete and partial responders to chemotherapy (9).

Chemoresponse is a significant outcome predictor in patients with head and neck cancer, regardless of the treatment modality used. The histoculture drug response assay (HDRA) has been shown to be a reliable method for in vitro chemoresponse assessment. In this study, we have correlated the HDRA assessment with survival in patients with head and neck squamous cell carcinoma (HNSCC). Tumor specimens from 41 of 42 patients undergoing treatment for HNSCC were successfully evaluated by the HDRA. Tumor tissue was histocultured on Gelfoam sponges gel in 24-well plates, followed by treatment with cisplatin (15 microg/mL) or 5-fluorouracil (40 microg/mL) in triplicate. A control group received no drug treatment. After completion of drug treatment, the relative cell survival in the tumors was determined using the MTT assay. The inhibition rate (IR) for each drug was calculated relative to the control for each case, and sensitivity was defined as a tumor IR of greater than 30%. Treatment was based on established protocols for the location and stage of the tumor and included surgery, radiation, and/or chemotherapy. Survival comparisons were performed using the generalized Wilcoxon test for the comparison of Kaplan-Meier survival curves. Resistance to 5-fluorouracil was present in 13 cases (32%), to cisplatinum in 13 cases (32%), and to both agents in 11 cases (27%). The 2-year cause-specific survival was significantly greater for patients sensitive to 5-fluorouracil (85% vs 64%; p=0.04), cisplatinum (86% vs 64%; p =0.05), or both agents (85% vs 63%; p =0.01). The association between HDRA assessment of chemoresponse and clinical outcome remained significant even after controlling for the effects of TNM stage and the presence of recurrent cancer at presentation by multivariate analysis. Chemosensitivity determined by the HDRA seems to be a strong predictor of survival in patients with advanced HNSCC and should be considered further (10).

A study to prospectively correlate clinical outcomes of advanced epithelial ovarian cancer (AEOC), with the results of in vitro chemosensitivity testing of paclitaxel and carboplatinum using the HDRA was performed. A total of 104 patients with AEOC were treated with combination chemotherapy of paclitaxel and carboplatinum after primary cytoreductive surgery between 2007 and 2012 at the Asan Medical Center, Seoul, Korea. To compare chemosensitivity in the HDRA with clinical response, all patients were first categorized into two groups as either sensitive to both paclitaxel and carboplatinum (SS), or not sensitive to one or both drugs (R) based on HDRA results. The recurrence rate was much lower in the SS group compared to the R group; 29.2% vs 69.8%, respectively (p=0.02). The SS group had a significantly longer progression-free survival compared to the R group, 34.0 months vs 16.0 months, respectively (p=0.025). These results demonstrate that the HDRA prospectively correlates to clinical outcome with AEOC from chemotherapy and that treatment regimens can be individualized based on the HDRA (11).

Lymph node metastasis is often the first indication of the aggressiveness of breast cancer. Effective chemotherapy in breast cancer depends on targeting the metastatic component of the disease. In order to optimize chemotherapy in the metastatic target of breast cancer, the HDRA was performed on surgical specimens of primary tumor and axillary lymph node metastasis from 30 breast cancer patients. The surgical specimens were cut into approximately 10 mg pieces, and placed onto the collagen gel sponges in the medium containing previously-determined cutoff concentrations of doxorubicin (DOX), 5-fluorouracil (5-FU), cisplatinum (CDDP), and mitomycin C (MMC). After incubation for 7 days, the chemosensitivity of the tumor fragments was evaluated with the MTT endpoint. The lymph node metastases were more resistant than the primary tumor for DOX, 5-FU, and MMC (p<0.05) but not for CDDP. The data suggest that both primary tumor and metastases from individual patients should be tested in the HDRA to enhance clinical efficacy of chemotherapy (12).

A high-throughput robotic tissue handler (“RTH”) is used, in some embodiments, to perform the HDRA. In some embodiments, the RTH includes the following:
a. Automated arm with a small bore 0.3-3 mm needle shaped tip that samples tissue cores
b. Tissue holder that immobilizes tissue in a physiological solution with an open top
c. An automated controller that directs the arm to sample the tissue at approximately 1-2 mm³ and ejects the tissue into wells of micro-well tissue culture plates
d. An automated stage that holds multi-well plates pre-filled with culture medium, and different chemotherapy drugs by the RTH, that moves such that the automated arm ejects sampled tissue into each well. After a period of incubation, the plates with tumor tissue are placed back on the automated stage.
e. The robotic arm is fitted with a small-bore vacuum device with a filter such that only the medium is removed from each well.
f. The robotic arm then delivers a colormetric or a fluorometric solution such as 3-(4,5-dimethylthiazol-2-yl)-2,5-diphenyltetrazolium bromide (MTT).
g. After a period of incubation, the optical density or fluorescence of each well is automatically determined on the robotic cell handler.
h. The fluorometric or optical density data are exported to a computer which calculates the response of each well to the drug in the well compared to wells with no drug. A report is then issued to the doctor on a scale of least effective to most effective drug for the patient's tumors.

A 360-well culture dish is used, in one non-limiting example. A total of 35 drugs are tested with 10-well replicates and 10-wells with no drug. Each well contains 1 mm³ tissue cube. Approximately 1 mg per well is sampled from 0.5 g of tumor tissue.

Example: A 360-well dish that contains 35 different drugs with 10 well receptacles for each drug and 10 wells with no drug. The robotic tissue handler will allocate 1 mm³ tumor fragments to each well and the drugs and incubate the 36 well plates at 37° C. for 5 days and then remove drug and medium from each well and replace with MMT solution for 13 hours and then read the optical density of each plate.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

What is claimed is:

1. A method for determining cancer treatment comprising steps:
obtaining cells from a tumor of a patient with a cancer;
establishing a three-dimensional in-vitro histoculture of the cells;
adding a drug to the histoculture; and
measuring a response of the histoculture to the drug.

2. The method of claim 1, wherein the drug is mitomycin C.

3. The method of claim 1, wherein the drug is doxorubicin.

4. The method of claim 1, wherein the drug is 5-flourouracil.

5. The method of claim 1, wherein the drug is cisplatin.

6. The method of claim 1, wherein the cancer is gastric cancer.

7. The method of claim 1, wherein the cancer is breast cancer.

8. The method of claim 1, wherein the cancer is colorectal cancer.

9. The method of claim 1, wherein the cancer is epithelial ovarian cancer.

10. The method of claim 1, wherein the cancer is squamous cell head and neck cancer.

11. The method of claim 1, wherein the establishing, adding, and measuring steps are performed by a robotic tissue handler.

12. A robotic tissue handler histoculture drug response assay comprising steps:
robotically obtaining a plurality of tissue cores from a tumor of a patient with a cancer;
placing one each of the plurality of tissue cores into each of a corresponding plurality of wells disposed on a controlled moving stage;
adding a mixture of a drug and a culture medium to each of the plurality of wells;
incubating the plurality of wells;
removing the mixture from each of the plurality of wells without removing the tissue cores;
adding a cell viability measuring solution to each of the plurality of wells;
reading cell viability of the plurality of tissue cores; and
correlating the cell viability with a resistance to the drug with an accuracy of at least about seventy-five percent (75%).

13. The assay of claim 12, wherein the drug is selected from the group of drugs consisting of mitomycin C, doxorubicin, 5-flourouracil, and cisplatin.

14. The assay of claim 12, wherein the cancer is gastric cancer.

15. The assay of claim 12, wherein the cancer is breast cancer.

16. The assay of claim 12, wherein the cancer is colorectal cancer.

17. The assay of claim 12, wherein the cancer is epithelial ovarian cancer.

18. The assay of claim 12, wherein the cancer is squamous cell head and neck cancer.

19. The assay of claim 12, wherein the cancer is a sarcoma.
